(19) United States
(12) Patent Application Publication
(10) Pub. No.: US 2013/0102945 A1
(43) Pub. Date: Apr. 25, 2013

(54) PROTECTIVE WOUND SHIELD

(71) Applicant: Tammy Hines Long, Fort Worth, TX (US)

(72) Inventor: Tammy Hines Long, Fort Worth, TX (US)

(73) Assignee: MARTIN LONG MEDICAL PRODUCTS, LLC, Colleyville, TX (US)

(21) Appl. No.: 13/808,568

(22) PCT Filed: Jul. 12, 2011
(86) PCT No.: PCT/US11/43730

§ 371 Date: Jan. 4, 2013

Related U.S. Application Data
(60) Provisional application No. 61/363,437, filed on Jul. 12, 2010.

(51) Int. Cl. A61F 13/02; A61M 25/02

(52) U.S. Cl. CPC A61F 13/0243; A61M 25/02
USPC 602/43; 604/180

(57) ABSTRACT

A light-weight, semi-rigid wound shield protects wounds and intradermal therapy ports from impacts between therapy sessions. The wound shield comprises a domed cover forming a concave cavity above the wound or therapy port. A flange surrounding the cavity presses against the patient's skin to deflect and distribute the force of impacts. The flange may include a self-adhesive layer on its bottom, or it may be secured with medical tape or a fitted bandage that overlaps the flange and the surrounding skin. A peel-away membrane covers the adhesive layers. A tunnel through the flange may connect to the cavity to accommodate a therapy tube which remains in place while the shield is worn. In an alternate embodiment, a base coextensive with the flange adheres to the patient's skin and the wound shield removably couples to the base by its flange. The assembly comes in a sterile envelope or package.

### BACKGROUND OF THE INVENTION

1. Field of the Invention

This invention relates generally to medical wound dressings, and particularly to wound dressings for covering intradermal chemotherapy and other treatment ports adapted for periodic reuse. More particularly, this invention relates to a flanged, rigid or semi-rigid, domed cover adapted to protect such ports or wounds from impact.

2. Description of Related Art

Chemotherapy and other ongoing treatment patients often have intradermal access ports installed to permit periodic injections of therapeutic treatment fluids. These can be very sensitive, and impacts upon them, even slight, can be very painful. Especially with physically active patients, or with access ports located in certain places on patients' bodies, the likelihood of an impact event is high. A need exists for a sterile protective cover which shields such intradermal treatment ports from most impacts.

Some wounds also can be very sensitive while healing. The typical gauze bandage, while effective for keeping such wounds sanitary and sterile, provides little or no impact resistance. A need exists for a protective cover for shielding wounds from impacts.

### SUMMARY OF THE INVENTION

A light-weight, rigid or semi-rigid wound shield protects wounds and intradermal therapy ports from painful and potentially injurious impacts while the patient goes about his daily life between therapy sessions. The wound shield comprises a truncated-dome-shaped cover that forms a concave cavity above the wound or therapy port. A flange surrounding the cavity presses against the patient's skin to deflect and distribute the force of impacts. The flange may include an adhesive layer on its bottom side that renders the wound shield self-adhesive. Additionally, or alternately, the wound shield may be secured with medical tape or with a fitted, adhesive web disposed atop the flange and overlapping onto the surrounding skin. A peel-away membrane covers the adhesive layers. A tunnel through the flange may connect to the cavity to accommodate a therapy tube which remains in place while the shield is worn. In an alternate embodiment, a base coextensive with the flange adheres to the patient's skin and the wound shield removably couples to the base by its flange. The assembly comes in a sterile envelope or package.

### BRIEF DESCRIPTION OF THE DRAWINGS

The novel features believed characteristic of the present invention may be set forth in appended claims. The invention itself, however, as well as a preferred mode of use and further objects and advantages thereof, will best be understood by reference to the following detailed description of an illustrative embodiment when read in conjunction with the accompanying drawings, wherein:

FIG. 1 shows in perspective view a preferred embodiment of the protective wound shield of the present invention in place on the shoulder of a patient.

FIGS. 2-4 depict in top plan, front and right side elevation views, respectively, of the preferred embodiment of the invention as in FIG. 1, including a tube tunnel through the planar flange surrounding the dome.

FIGS. 5 and 6 show top plan and elevation views, respectively, of a circular, first alternate embodiment of the present invention having no tube tunnel.

FIGS. 6A-6C show perspective and front and side elevational views, respectively, of an embodiment of the present invention having a curved flange.

FIGS. 7 and 8 depict in perspective views an oval-shaped, second alternate embodiment, and a rectangular-shaped, third alternate embodiment, respectively, of the present invention.

FIGS. 9A, 9B depict in plan and partial cross section views respectively the second alternate embodiment of FIG. 7 bearing an adhesive layer surmounting the bottom of, and co-extensive with, the flange, including a peel-away layer for protecting same while in storage.

FIG. 10A, 10B depict in plan and partial cross section views respectively the preferred embodiment of FIG. 1 bearing a self-adhesive bandage surrounding and surmounting the flange.

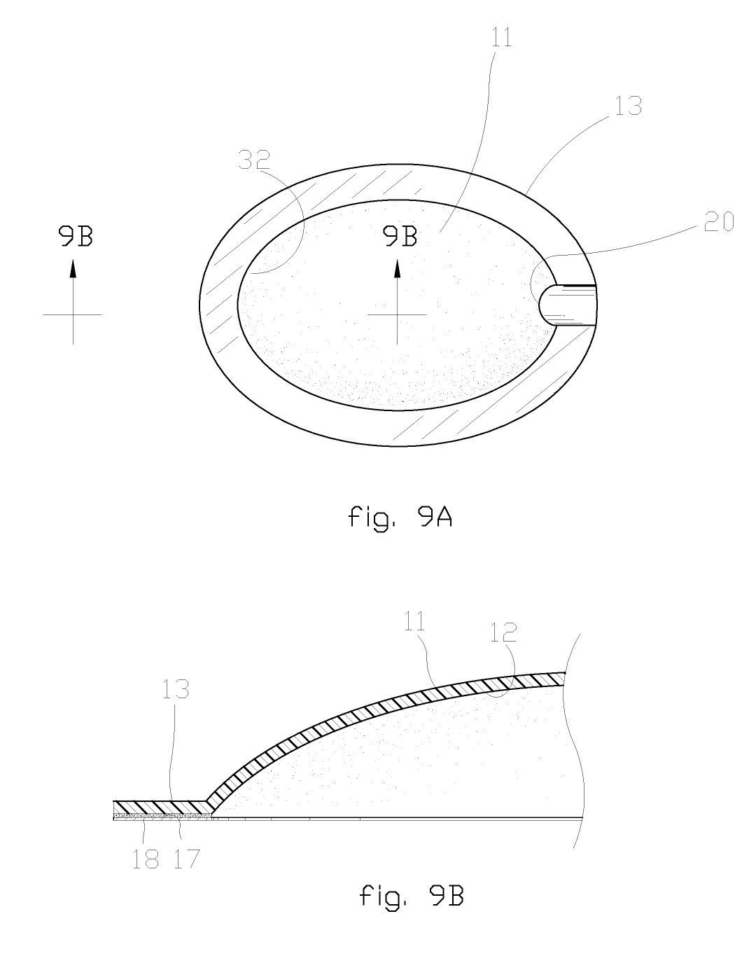

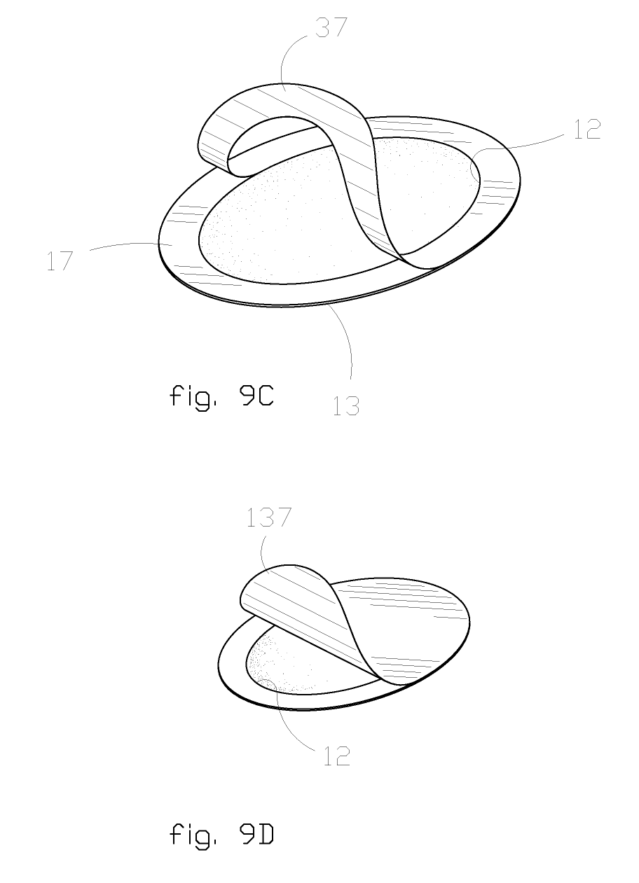

FIGS. 9C, 11 show in bottom perspective view the preferred and second alternate embodiments of the present invention with a peel-away membrane covering and protecting the bottoms of the provided adhesive layers while the invention is in storage.

FIG. 9D, 12 depict in bottom perspective views the preferred and second alternate embodiments of the peel-away layers of FIGS. 9C, 11 wherein the peel-away layers cover the dome cavities as well.

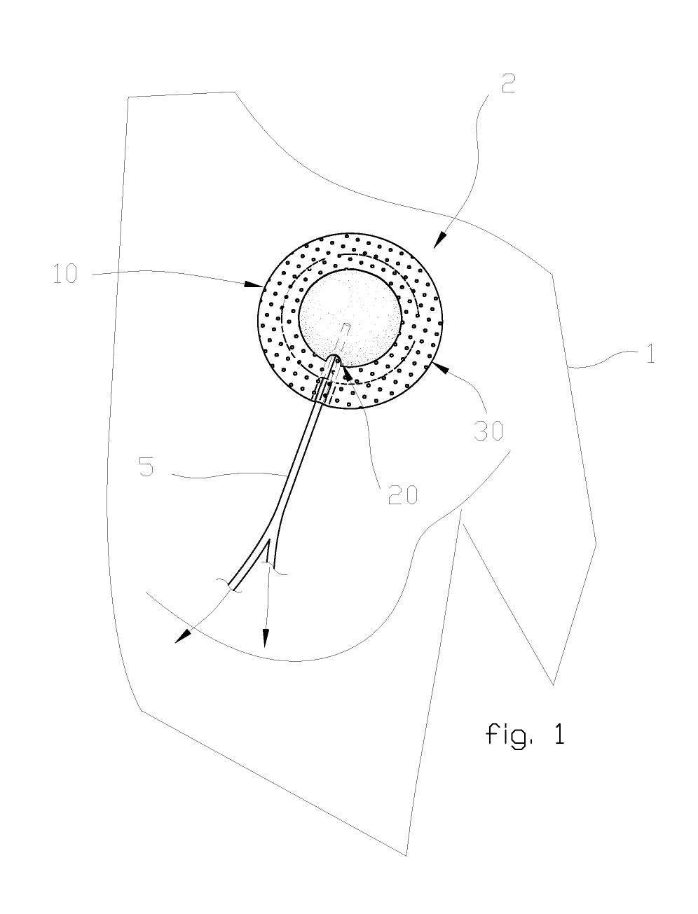

FIG. 13 shows the preferred embodiment of FIG. 1, bearing the self-adhesive bandage as shown in FIG. 9, enclosed in a sterile envelope packaging.

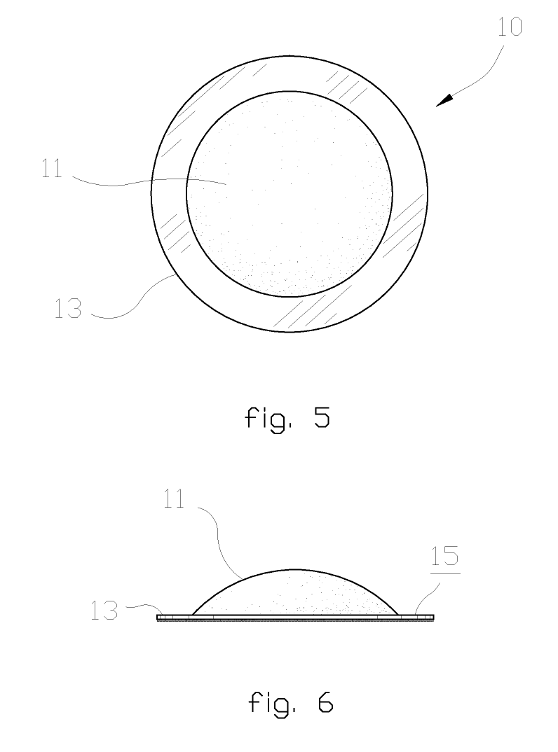

FIG. 14 shows another alternate embodiment of the present invention having a base that adheres to a patient's skin and surmounted by a flanged dome similar to that of FIG. 5 coupled to the base by a flexible hinge.

FIG. 15 depicts the alternate embodiment of FIG. 14 in plan view

FIG. 16 details in elevational cross section as indicated in FIG. 15 the alternate embodiment of FIG. 14, showing the base and adhesive layers of this embodiment.

### DESCRIPTION OF A PREFERRED EMBODIMENT

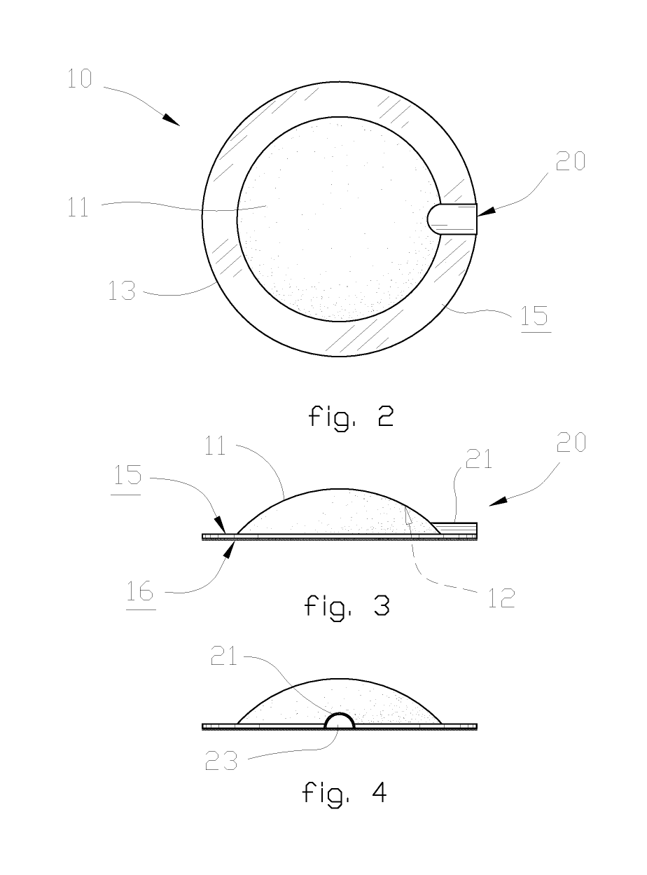

Referring now to FIGS. 1-6, shield 10 of the present invention is shown in FIG. 1 in place on site 2, in this case the chest, of user patient 1 and held in place by circular bandage 30 Protruding from beneath bandage 30, tube 5 extends through tunnel 20 to beneath shield 10 where it may connect to a therapy port (not shown).

Shield 10 comprises a dome 11 forming upwardly concave cavity 12 above site 2. Cavity 12 at its apex preferably rises at least one-half (½ in.) inch above the plane of site 2 and provides space into which may extend any therapy port couplings, bandages or other objects (not shown) associated therewith. Dome 11 thereby provides a protective roof over site 2 adapted to reduce or eliminate contact with site 2 by clothing, stray hands or unexpected impacts which may impinge upon site 2.

Surrounding and extending radially outward from dome 11, flange 13 integrates with dome 11 at its perimeter to provide a widened engagement surface for shield 10 to bear upon the skin of patient 1 in the vicinity surrounding site 2. For most applications, flange 13 preferably measures approximately one-half (½ in.) inch in width, but one having ordinary skill in the art will recognize that the width of flange 13 may vary for larger and smaller shields 10.

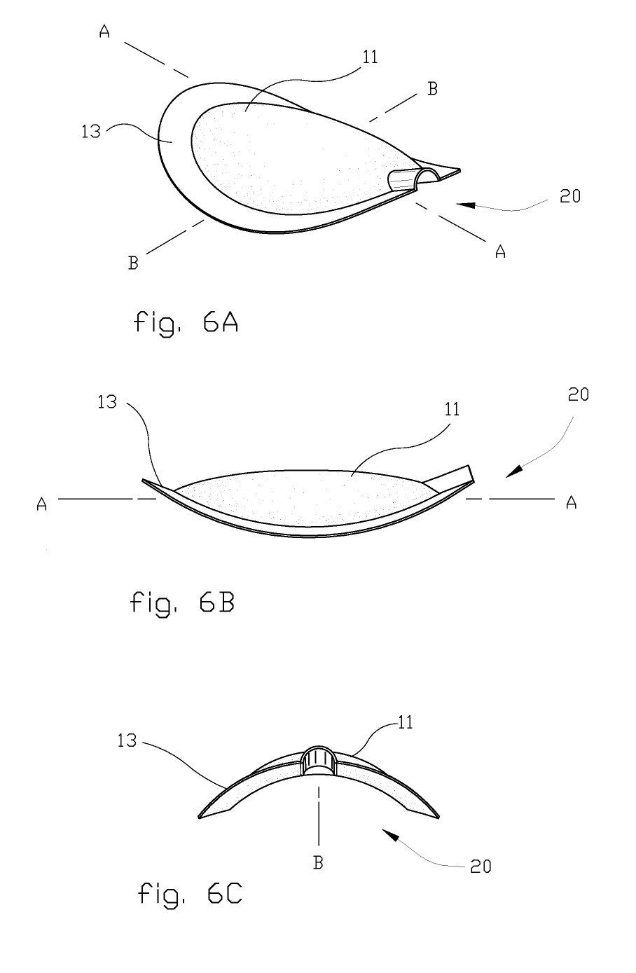

Further, preferably and for most applications, flange 13 is substantially flat, occupying a single plane. One having ordinary skill in the art will recognize, however, that for specific applications, such as on patient 1's leg or another more curved body site 2 (not shown), flange 13 may be curved along any major or minor axis to match more accurately the curvature of said other body site 2. For example, FIGS. 6A-6C depict an embodiment of the invention wherein flange 13 is convexly curved along its major axis A while concavely curved along is transverse minor axis B. Such a configuration would work well on a patient's arm (not shown) which may not be flat enough where wound site 2 is located.

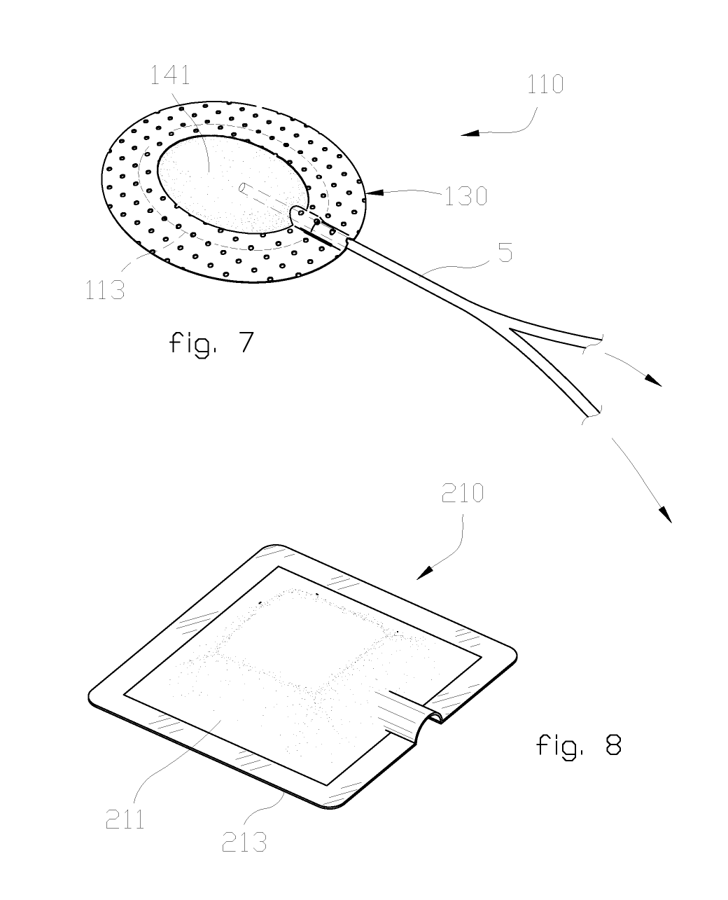

In FIGS. 1-6, shield 10 is depicted as being substantially round, and dome 11 is represented as a sphere truncated by the plane occupied by flange 13. One having ordinary skill in the art will recognize, however, that shield 10 may comprise any number of other shapes, such as oval 110 or square 210 (see FIGS. 7-9) without departing from the spirit and scope of the present invention. In such case, flanges 113, 213, respectively, will match the shape of the perimeter of domes 111, 211 respectively.

Similarly, one having ordinary skill in the art will recognize that shields 10, 110, 210 may be any size necessary to cover site 2 sufficiently to protect the wound or therapy port (neither shown) therein. Preferably, shield 10, 110, 210 for most applications will be approximately five (5 in.) inches in diameter, including flange 13, 113, 213. Where larger or smaller domes 11, 111, 211 are needed for different applications, flange 13, 113, 213 preferably will remain approximately one-half (½ in.) inch wide surrounding the perimeter of dome 11, 111, 211.

NOTE: henceforth herein, all shapes of shields considered to be in conformity with the present invention will be referred to using reference numerals for the preferred embodiment of shield 10, including all corresponding components thereof bearing other reference numbers. Only where the particular shape of an alternate embodiment such as shields 110, 210 requires it will particular references to those embodiments or components thereof be used. One having ordinary skill in the art will recognize that such discussions apply equally to each of such alternate embodiments absent specific indication to the contrary.

As shown in FIGS. 1-4, and 7-10, tunnel 20 disposed in at least one location around the perimeter of dome 11 provides access for tube 5 through flange 13 into cavity 12. One having ordinary skill in the art will recognize that, especially in the case of therapy ports (not shown), tubing 5 sometimes must remain in place while patient 1 goes about daily activities between or even during therapy sessions. For example, sometimes chemical therapy fluids periodically are injected into said therapy port throughout the day from a source therefor, such as a portable machine (not shown) carried or worn by patient 1. In other cases, tubing 5 may be left in place and strapped to the torso of patient 1 for subsequent therapy treatments from fixed machinery or facilities (neither shown). In all such cases, tubing 5 remains in place, extending from said therapy port beneath cavity 12, and is secured in place on patient 1's torso with medical tape or other means (neither shown) so it won't move and irritate site 2.

As depicted in FIGS. 5-6, shield 10 is surrounded by continuous flange 13 which has no tunnel 20 interrupting flange 13. Shield 10 merely covers site 2 without providing access to cavity 12. Such a configuration might be used, for example, for covering of simple wounds which, though they may be particularly painful or sensitive, do not require leaving tubing 5 in place. In such cases, cavity 12 serves as described above to ward off undesired contact by clothing or other unwanted impacts. In other applications, however, where tubing 5 must be left in place, an embodiment of shield 10 having tunnel 20 will prove more useful. One having ordinary skill in the art also will recognize, however, that shield 10 as depicted in FIGS. 5, 6 and lacking tunnel 20 also could be used with tubing 5 by simply running tubing 5 beneath flange 13. This arrangement, however, would risk collapsing tube 5 under flange 13 and could cause flange 13 to be disengaged from patient 1's skin for a spaced distance on either side of tubing 5, requiring further taping or other sealing measures to assure the sterility integrity of cavity 12.

Tunnel 20 preferably is formed as a downwardly concave channel open at its bottom side flush with bottom 15 of flange 13. Tunnel 20 extends radially outward from cavity 12 to the perimeter of flange 13, thereby forming a gap in flange 13 wide enough for tubing 5 to fit through. Arch 21 formed by tunnel 20 necessarily extends above flange 13 to intersect dome 11 radially inward toward the center of shield 10 to the extent that curvature of dome 11 requires. Arch 21 integrates seamlessly with dome 11 at such intersection point to assure the integrity of cavity 12.

The shape of arch 21 and tunnel 20 may vary. For example, arch 21 may be a full semicircle having a radius equal to twice the outside diameter of tubing 5. This permits tubing 5 and the bottom of flange 13 to lie flush against the skin of patient 1 and keeps flange 13 fully engaged with said skin without tubing 5 depressing it appreciably. Such arrangement in some cases may be important for deterring discomfort to patient 1. One having ordinary skill in the art will recognize, however, that this arrangement leaves a peripheral gap on either side of tubing 5 between it and the widest part of arch 21 and tunnel 20. Such peripheral gap compromises the integrity of cavity 12, and if the sterile integrity of cavity 12 is to be maintained, must be sealed with medical tape by a nurse (not shown) or other user such as patient 1.

Alternately, arch 21 may have a radius substantially equal to the outside diameter of tubing 5, but be disposed above flange 13 by the same distance, the resulting vertical separation being closed by vertical tunnel walls (not shown) extending downward from arch 21 to intersect flange 13. This configuration greatly decreases, but does not eliminate, the peripheral gap between tube 5 and tunnel 20, thus still requiring taping, though perhaps not as much. The benefit of such arrangement is that tubing 5 and flange 13 remain flush with the skin of patient 1, maximizing closure of cavity 12 even without using medical tape.

As another alternate, tunnel 20 could be integral with or disposed above flange 13 (neither shown) while of appropriate diameter for tubing 5 to be inserted through tunnel 20 from within cavity 12 before flange 13 is engaged with patient 1's skin. Still further, tunnel 20 could comprise a short segment of tubing affixed to flange 13 and extending into cavity 12 to couple to therapy port couplings (not shown). Such a tunnel 20 further could have a nozzle (not shown) extending outward beyond the radius of flange 13 to provide a place to which tubing 5 may be coupled. One having ordinary skill in the art will recognize that all such configurations for tunnel 20 are considered to be within the spirit and scope of the present invention.

In any shape, size or tunnel 20 configuration, shield 10 must adhere to site 2 so that it doesn't slip off and therefore fail to provide the protection for which it is intended. This may be accomplished by simply taping shield 10 to site 2 using strips of any of a number of readily available medical tapes adapted to secure bandages to patient 1's skin. A plurality of said strips of tape (not shown) would be placed in chordal positions around the perimeter of flange 13 and overlapping onto the skin of patient 1 in the vicinity of site 2. The width and type of said tape so employed is not of particular specification, except that it preferably comprises a standard medical tape of appropriate size and adapted to adhere, but be readily removable from without causing injury, to the skin of patient 1.

Turning now also to FIGS. 9A-9D, an alternative to said medical tape appears. Disposed beneath flange 13 such that it would engage the skin of patient 1 when flange 13 is placed around site 2, flange adhesive 17 surrounds the perimeter of dome 11 substantially coextensive with flange 13. Thus equipped, shield 10 may be used alone as a self-adhesive protection for site 2. Peel-away membrane 18 protects flange adhesive 17 from contamination and retains its adhesive quality until used at site 2, as discussed in more detail below for the preferred embodiment employing bandage 30.

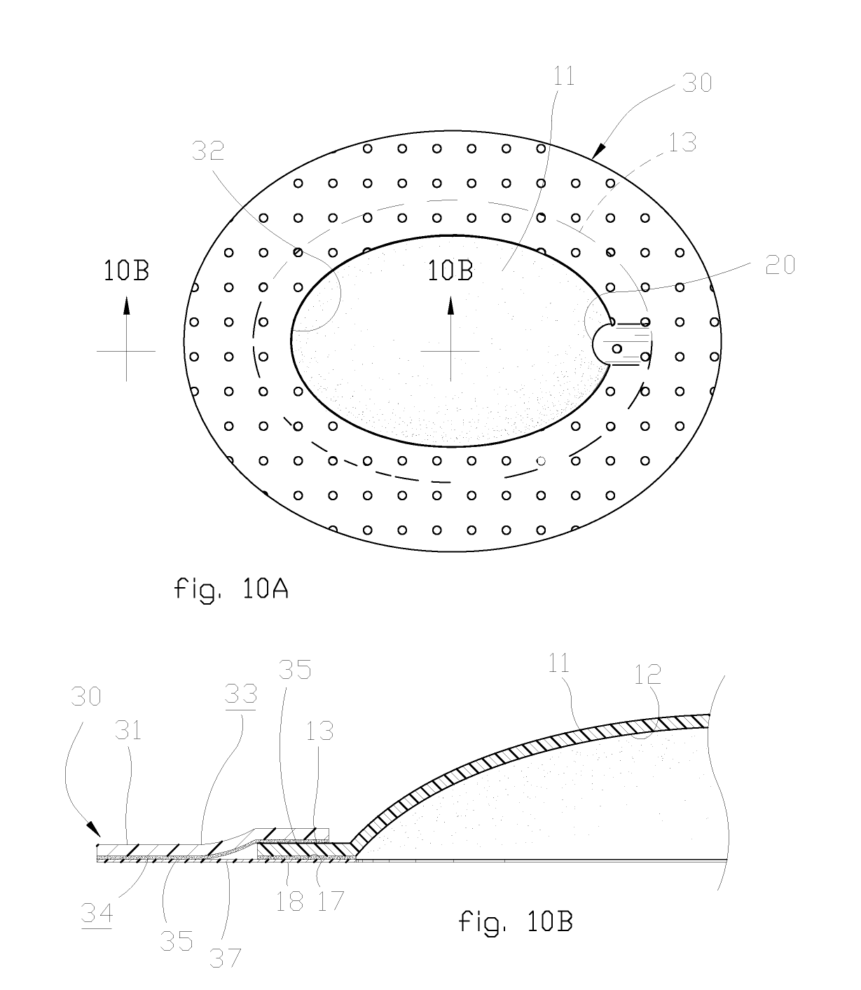

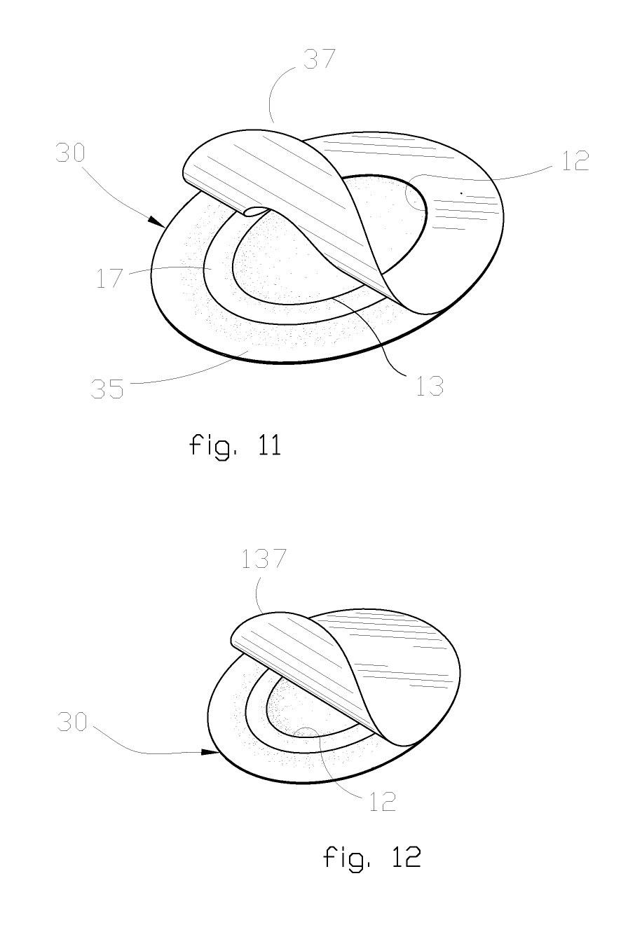

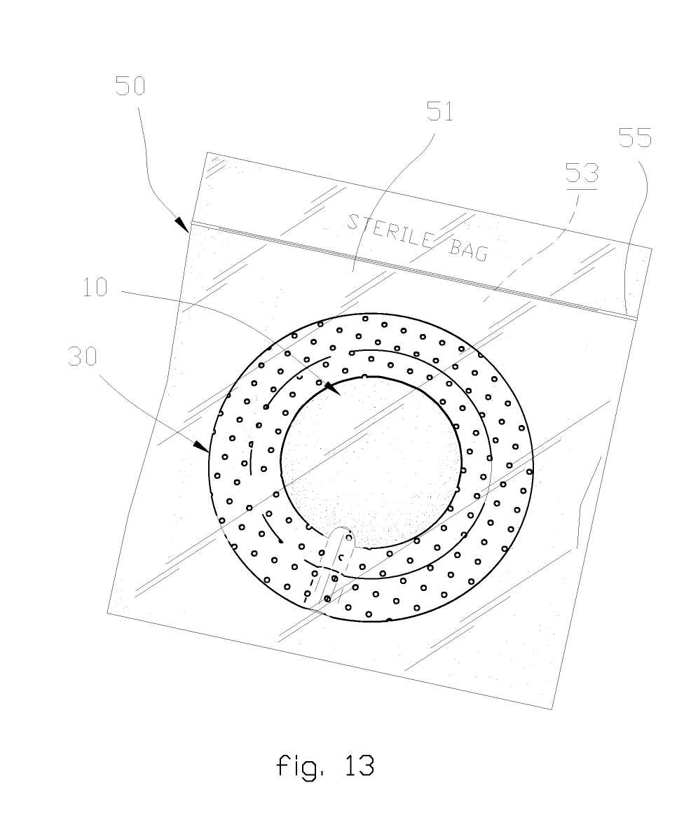

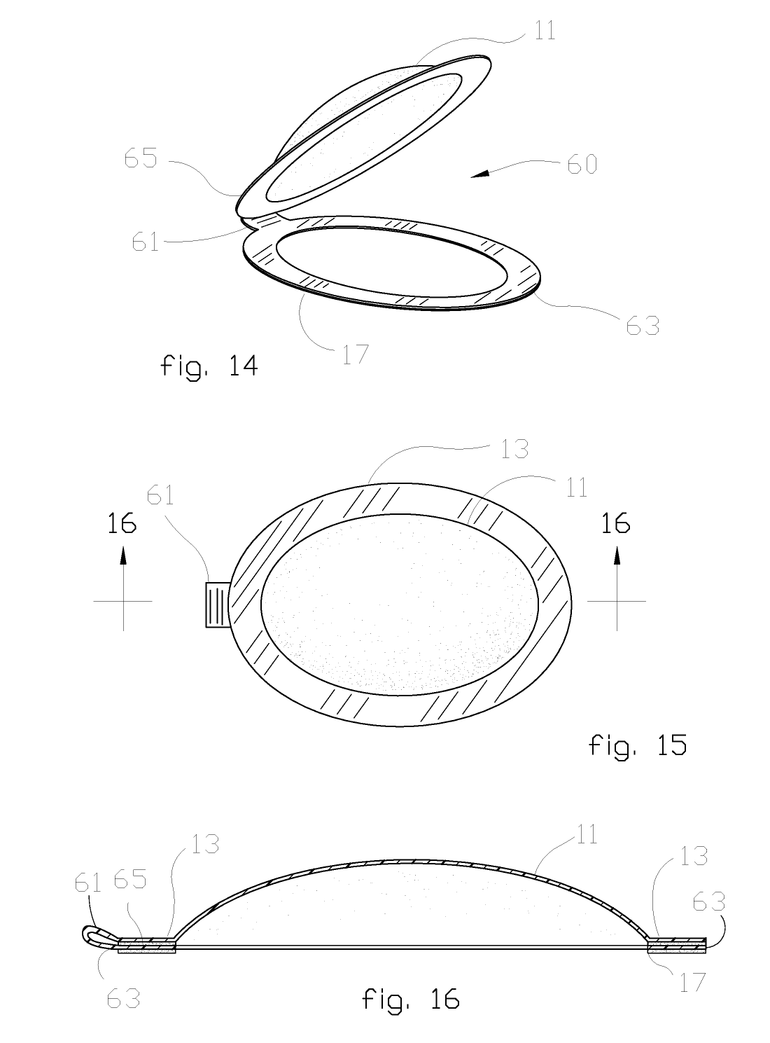

Another optional alternative to medical tape depicted in FIGS. 10A-12 comprises bandage 30 disposed atop flange 13 and overlapping onto patient 1's skin to secure shield 10 in place around site 2. Preferably, bandage 30 comprises a flexible web 31 of a suitable plastic or fabric material shaped to the size and shape of flange 13. Where flange 13 is round, bandage 30 has an inside diameter substantially equal to the inner diameter of flange 13 such that bandage 30 lies juxtaposed to dome 11 around its entire perimeter but leaving dome 11 uncovered. The outside perimeter of bandage 30 extends beyond the outside perimeter of flange 13 by at least the width of flange 13, thus overlapping onto the skin of patient 1 by that amount. Bandage adhesive 35 coats the entire bottom surface 34 of web 31 such that, where it is disposed atop flange 13, it adheres thereto to hold bandage 30 securely onto shield 10. The portion of bandage 30 extending beyond flange 13 engages patient 1's skin with bandage adhesive 35 to secure the whole device in place above site 2.

Peel-away protective layer 37 disposed on the under side of bandage 30 keeps adhesive layers 17, 35 unexposed until such time as bandage 30 is needed. Protective layer 37 could take several forms. Where shield 10 is provided with no adhesive means 17, or where only flange adhesive 17 is provided thereon, bandage 30 could be provided separately as an optional alternative to medical tape. In such case, protective layer 37 preferably would be coextensive with and would line the bottom of bandage 30. Alternately, where shield 10 is provided with bandage 30 already installed (as in FIGS. 1, 7, 10; see also FIG. 13 discussed in more detail below), flange adhesive 17 may or may not be employed at all. Where flange adhesive 17 is present, peel-away protective layer 37 may extend only over the portion of web 31 outside the perimeter of flange 13, with flange adhesive protective layer 18 covering flange adhesive 17. Preferably in such case, however, protective layer 37 extends across both bandage adhesive 35 and flange adhesive 18 in a continuous lamination, as depicted in FIGS. 11 and 12, that easily may be removed with one motion. As shown in FIG. 11, such peel-away layer 37 is coextensive with bandage 30 and flange 30, but does not cover cavity 12. Alternatively, peel-away layer 37 could cover cavity 12, as shown in FIG. 12, thereby further assuring the sterile integrity of cavity 12 while shield 10 is being stored. One having ordinary skill in the art will recognize that all such configurations are considered to be within the spirit and scope of the present invention.

Turning now also to FIG. 13, packaging for shield 10 preferably comprises a sterile envelope adapted to contain a single shield 10. Package 50 comprises two, preferably coextensive, laminations 51, 53 of a suitable web material adapted to be sealed together around their mutual perimeters to enclose entirely one shield 10. Preferably, at least one of said laminations 51, 53 comprises a transparent material through which shield 10 is visible. Package 50 may include resealing means 55 which permits opening and closing package 50, but preferably, package 50 provides a sterile storage environment for shield 10 which is opened only upon usage. One having ordinary skill in the art will recognize that all reasonable packaging arrangements, including bulk, unsterile packaging (not shown) is considered to be within the spirit and scope of the present invention.

Turning now to FIGS. 14-16, another optional feature of the present invention includes hinged protective cover 60 comprising base 63 adapted to adhere to patient 1's skin with adhesive layer 17, as discussed above for the preferred and alternate embodiments 10, 110, 210. Coupled to base 63 by hinge 61, flange 13 surrounding dome 11 is substantially coextensive with base 63 and is adapted to mate therewith and cover wound site 2, as described above. Second adhesive later 65 affixes the bottom of flange 13 to the top of base 63 to keep dome 11 in place while patient 1 goes about his daily life. Cover 60 provides means whereby site 2 may be repeatedly accessed without the need to completely remove the present invention and replace it each time. By lifting flange 13 away from base 63 at its edge opposite hinge 61, a user causes second adhesive layer 65 to disengage from base 63 and permit dome 11 and flange 13 to swing out of the way (as in FIG. 14). A tab (not shown) might be provided on flange 13 opposite hinge 61 to aid in separating flange 13 from base 63. Once the purpose for which access to site 1 is accomplished, simply pressing flange 13 against base 63 again causes second adhesive later 65 again to adhere and retain dome 11 over and protecting site 1.

Cover 60 has been discussed above as having hinge 61 and second adhesive layer 65 for accomplishing the desired result of making site 2 repeatedly accessible without having to reinstall cover 60 each time. One having ordinary skill in the art will recognize that other means could be employed to make dome 11 temporarily removable, such as replacing second adhesive layer 65 with a hook-and-loop layer (commonly known as Velcro), with or without eliminating hinge 61, without departing from the spirit and scope of this embodiment of the present invention, and that all such temporary adherence means are considered to be within the spirit and scope of the present invention.

Shield 10 preferably is fabricated from a rigid or semi-rigid thermoplastic material readily moldable through conventional injection molding processes into the desired shapes described above. More preferably, shield 10 is fabricated from a medical grade polyester which itself is capable of being sterilized and does not emit or sluff off its own contaminants which might derogate from the integrity of cavity 12. Such material preferably is approximately forty-three thousandths (0.043″) inches in thickness. Still more preferably, shield 10 is transparent or translucent to provide visible feedback while positioning shield 10 over site 2, as described below. A suitable polyester for shield 10 comprises model Prime PETG 6763, or PETG 14471, both available from Primex Plastics Corporation of Richmond, Ind., USA. Adhesives 17, 35 preferably are made from hypoallergenic medical adhesives designed for sustained contact with human skin, commonly used on bandages and medical tape. Likewise, bandage 30 may be made of a flexible polyethylene plastic or woven fabric commonly employed to fabricate such self-adhesive bandages. A suitable product for bandage 30, including adhesive 35, is available as Product No. DM-6001 from DermaMed Coatings Company, LLC, of Tallmadge, Ohio, USA. A suitable primary adhesive 17 for flange 13 and base 63 is available as Product No. 9943 from 3M Medical Specialties Company of St. Paul, Minn., USA. A suitable secondary adhesive 63 for interface between flange 13 and the top of base 63 is available as Product No. 9833 from 3M Medical Specialties Company of St. Paul, Minn., USA. Peel-away layers 37, 18 also preferably are fabricated from flexible, polyethylene plastic and fabricated in two parts to overlap one part over the other to provide convenient means for a user to remove them when needed.

In operation, shield 10 of suitable size and shape for application to site 2 is selected and removed from package 50. Peel away layer 37 is removed and cavity 12 inverted above site 2 so that all portions of flange 13 are disposed substantially equidistant from the wound or therapy port at site 2. Where tubing 5 must be left in place, it is taped into position extending from site 2. Then, a suitable shield 20 having tunnel 20 through flange 13 is selected and tunnel 20 is oriented to straddle tubing 5. Shield 10 then is secured in place by pressing it onto patient 1's skin until adhesive 35 bonds sufficiently therewith to hold shield 10 in place. If desired, the user may supplement bandage 30 and flange adhesive 17 with strips of medical tape to assure that shield 10 remains in place even under physical impacts from sports activities.

Thus, shield 10 as described herein in its preferred and alternate embodiments provides a light-weight, rigid or semi-rigid cover for wounds and therapy ports for patient 1 to employ while going about his daily life. Shield 10 prevents or dramatically softens impacts on or around site 2 and gives patient 1 a less stressful and less painful experience while undergoing therapy for which the patient needs the therapy port, or simply while a significant wound is healing.

While the invention has been particularly shown and described with reference to preferred and alternate embodiments, it will be understood by those skilled in the art that various changes in form and detail may be made therein without departing from the spirit and scope of the invention. For example, though shield 10 is described above as being transparent, it could be preferable in some cases that it be opaque to conceal the therapy port and/or wound. Also, bandage 30 has been described herein as surrounding dome 11 without covering it. However, bandage 30 could instead cover dome 11 partially or entirely (neither shown) so that shield 10 better resembles a conventional, self-adhesive bandage having perhaps a more conventional and desirable aesthetic appearance and at least partly concealing shield 10 and the therapy port or wound beneath it. Still further, bandage 30 has been described herein as continuous around the perimeter of dome 11, but instead it could comprise strips of bandage material (not shown) adapted to be placed across flange 13 in selected locations around dome 11 without entirely covering flange 13.

Dome 11 also has been discussed herein as continuous and serving among other things to seal off wound site 2 from environmental contaminants. Dome 11 could, however, include one or more breathing holes to allow evaporation of perspiration and to allow fresh air to reach wound site 2.

I claim:

1. A protective wound shield for covering a wound site on a patient's skin, said shield having a longitudinal major shield axis and a transverse minor shield axis and comprising
a flange having a flange bottom surface and an opposite flange top surface, said flange further having a flange outer perimeter surrounding a flange inner margin adapted to surround said wound site;
a concave dome having a dome margin coupled to said flange inner margin and spanning said wound site to define an interior shield cavity; and
adhesive means for adhering said wound shield to said patient's skin.

2. The protective wound shield of claim 1 wherein said adhesive means comprises
an adhesive layer disposed on at least a portion of said flange bottom surface.

3. The protective wound shield of claim 1 wherein said adhesive means comprises
a bandage adapted to at least partially surround said dome margin and to overlap said flange and said patient's skin, said bandage having a bandage lower surface bearing an adhesive layer adapted to adhere to both of said flange top surface and said patient's skin surrounding said flange outer perimeter.

4. The protective wound shield of claim 1 and further comprising
at least one tunnel means communicating between said inner shield cavity and said flange outer perimeter, said tunnel means adapted to admit medical tubing into said shield cavity said wound shield is in place over said wound site.

5. The protective wound shield of claim 4 wherein said tunnel means comprises
a portion of said flange deviating upward from said flange top surface to form an aperture extending inward beneath said flange and into said shield cavity, said aperture sized and adapted to receive snugly and to surround at least a portion of said medical tubing.

6. The protective wound shield of claim 4 wherein said tunnel means is disposed coaxial with one of said major or minor shield axes.

7. The protective wound shield of claim 1 wherein
said flange is convexly curved along at least one of said major and minor shield axes.

8. The protective wound shield of claim 1 wherein
said flange is concavely curved along at least one of said major and minor shield axes.

9. The protective wound shield of claim 1 wherein
said flange is convexly curved along one of said major and minor shield axes and concavely curved along another one of said major and minor shield axes.

10. The protective wound shield of claim 1 and further comprising
a base congruent with said flange and disposed between said flange and said patient's skin, said base having a base upper surface, a base lower surface and a base outer perimeter surrounding a base inner margin;
adhesive means for adhering said base to said patient's skin; and
closure means for closing said shield cavity by affixing said flange to said base.

11. The protective wound shield of claim 10 and further comprising
a hinge coupled between said base and said flange.

12. The protective wound shield of claim 10 wherein said closure means comprises
an adhesive lamination disposed on at least a portion of said flange bottom surface and adapted to removably adhere to a corresponding portion of said base upper surface.

13. The protective wound shield of claim 10 wherein said adhesive means comprises
a bandage adapted to at least partially surround said base inner margin and to overlap said base and said patient's skin, said bandage having a bandage lower surface bearing an adhesive layer adapted to adhere to both of said base upper surface and said patient's skin surrounding said base outer perimeter.

14. A protective shield for an intradermal access port site on a patient's skin, said shield having a longitudinal major shield axis and a transverse minor shield axis and comprising
a flange having a flange bottom surface and an opposite flange top surface, said flange further having a flange outer perimeter surrounding a flange inner margin adapted to surround said access port site;
a concave dome coupled to said flange inner margin and spanning said access port site to defining an interior shield cavity;
at least one tunnel communicating between said inner shield cavity and said flange outer perimeter, said tunnel adapted to receive medical tubing extending into said shield cavity to said access port site
an annular base disposed between said flange and said patient's skin, said base having a base upper surface, a base lower surface and a base outer perimeter surrounding a base inner margin;
adhesive means for adhering said base to said patient's skin;
a hinge coupled between said base and said flange; and
closure means for closing said shield cavity by affixing said flange to said base.

15. The protective shield of claim 14 wherein said closure means comprises
an adhesive layer disposed on at least a portion of said flange bottom surface and adapted to removably adhere to a corresponding portion of said base upper surface.

16. The protective shield of claim 14 wherein said adhesive means comprises
a bandage adapted to overlap said base and said patient's skin, said bandage having a lower surface bearing an adhesive layer adapted to affix said bandage to both of said base upper surface and said patient's skin surrounding said base outer perimeter.

17. The protective shield of claim 14 wherein
said flange and said base are curved congruently with each other and along at least one of said major and minor shield axes.

18. An improved method of protecting an intradermal access port site on a patient's skin, the intradermal access port adapted periodically to provide access through said patient' skin for therapy injections administered through medical tubing coupled to said access port site, the improved method comprising
providing a substantially rigid shield adapted to cover said access port site, said shield having
an annular flange sized to surround said access port site, said annular flange having a flange lower surface and a flange upper surface and a flange inner margin and a flange outer margin;
a concave dome coupled to said flange inner margin and spanning said access port site to define an interior shield cavity;
adhesive means for adhering said rigid shield to said patient's skin; then
placing said rigid shield atop said patient's skin with said annular flange surrounding said access port site; then
employing said adhesive means to affix said rigid shield to said patient's skin.

19. The improved method of claim 18 wherein
said rigid shield further comprises
at least one radially disposed tunnel communicating between said interior shield cavity and said flange outer margin, said tunnel adapted to receive said medical tubing extending into said shield cavity from outside said flange outer margin while said shield is in place over said access port site;
and wherein
said improved method includes the additional steps of
coupling said medical tubing to said intradermal access port site; and
extending said medical tubing through said radially disposed tunnel

20. The improved method of claim 18 wherein
said rigid shield further comprises
an annular base substantially congruent with said annular flange and having a base top surface and a base bottom surface, said annular base adapted to be disposed between said annular flange and said patient's skin;
a hinge coupled between said annular base and said annular flange; and
an adhesive layer disposed between said base top surface base and said flange lower surface, said adhesive layer adapted to remobably affix said annular flange to said annular base;
and wherein
said improved method includes the additional steps following the employing step of repeatedly
urging said annular flange upward from said base top surface until said adhesive layer releases said annular flange to provide temporary access to said interior shield cavity; then
urging said annular flange back atop said annular base to cause said adhesive layer again to removably affix said annular flange to said annular base, thereby resealing said interior shield cavity.
